(19) United States
(12) Patent Application Publication
(10) Pub. No.: US 2024/0335280 A1
(43) Pub. Date: Oct. 10, 2024

(54) PROSTHETIC HEART VALVE STENT DEFLECTION

(71) Applicant: St. Jude Medical, Cardiology Division, Inc., St. Paul, MN (US)

(72) Inventors: Peter J. Ness, Minneapolis, MN (US); Abhijeet Joshi, Irvine, CA (US); Victoria Schuman, Minneapolis, MN (US); Jaron J. Olsoe, Minneapolis, MN (US)

(73) Assignee: St. Jude Medical, Cardiology Division, Inc., St. Paul, MN (US)

(21) Appl. No.: 18/589,676

(22) Filed: Feb. 28, 2024

Related U.S. Application Data
(60) Provisional application No. 63/494,270, filed on Apr. 5, 2023.

(51) Int. Cl. A61F 2/24

(52) U.S. Cl. CPC A61F 2/2415; A61F 2/2418; A61F 2210/0057; A61F 2220/0058; A61F 2230/0054; A61F 2250/001; A61F 2250/0018; A61F 2250/0036

(57) ABSTRACT

A prosthetic heart valve includes a collapsible and expandable stent having an inflow end and an outflow end, and a plurality of struts defining a plurality of cells, the stent being formed of a first material, a plurality of commissure attachment features formed of a second material that is different than the first material of the stent, and a collapsible and expandable valve assembly including a plurality of leaflets connected to the plurality of commissure attachment features.

### CROSS-REFERENCE TO RELATED APPLICATIONS

This application claims the benefit of the filing date of U.S. Provisional Patent Application No. 63/494,270, filed Apr. 5, 2023, the disclosure of which is hereby incorporated by reference herein.

### BACKGROUND OF THE DISCLOSURE

Valvular heart disease, and specifically aortic and mitral valve disease, is a significant health issue in the United States. Valve replacement is one option for treating heart valve diseases. Prosthetic heart valves, including surgical heart valves and collapsible/expandable heart valves intended for transcatheter aortic valve implantation or replacement (“TAVI” or “TAVR”) or transcatheter mitral valve replacement (“TMVR”), are well known in the patent literature. Surgical or mechanical heart valves may be sutured into a native annulus of a patient during an open-heart surgical procedure, for example. Collapsible/expandable heart valves may be delivered into a patient via a tube-like delivery apparatus such as a catheter, a trocar, a laparoscopic instrument, or the like to avoid a more invasive procedure such as full open-chest, open-heart surgery. As used herein, reference to a “collapsible/expandable” heart valve includes heart valves that are formed with a small cross-section that enables them to be delivered into a patient through a tube-like delivery apparatus in a minimally invasive procedure, and then expanded to an operable state once in place, as well as heart valves that, after construction, are first collapsed to a small cross-section for delivery into a patient and then expanded to an operable size once in place in the valve annulus.

Collapsible/expandable prosthetic heart valves typically take the form of a one-way valve structure (often referred to herein as a valve assembly) mounted to/within an expandable stent. In general, these collapsible/expandable heart valves include a self-expanding or balloon-expandable stent, often made of nitinol or another shape-memory metal or metal alloy (for self-expanding stents) or steel or cobalt chromium (for balloon-expandable stents). Existing collapsible/expandable TAVR devices have been known to use different configurations of stent layouts—including straight vertical struts connected by “V”s as illustrated in U.S. Pat. No. 8,454,685, or diamond-shaped cell layouts as illustrated in U.S. Pat. No. 9,326,856, both of which are hereby incorporated herein by reference. The one-way valve assembly mounted to/within the stent includes one or more leaflets and may also include a cuff or skirt. The cuff may be disposed on the stent's interior or luminal surface, its exterior or abluminal surface, and/or on both surfaces. A cuff helps to ensure that blood does not just flow around the valve leaflets if the valve or valve assembly is not optimally seated in a valve annulus. A cuff, or a portion of a cuff disposed on the exterior of the stent, can help retard leakage around the outside of the valve (the latter known as paravalvular or “PV” leakage).

Balloon expandable valves are typically delivered to the native annulus while collapsed (or “crimped”) onto a deflated balloon of a balloon catheter, with the collapsed valve being either covered or uncovered by an overlying sheath. Once the crimped prosthetic heart valve is positioned within the annulus of the native heart valve that is being replaced, the balloon is inflated to force the balloon-expandable valve to transition from the collapsed or crimped condition into an expanded or deployed condition, with the prosthetic heart valve tending to remain in the shape into which it is expanded by the balloon. Typically, when the position of the collapsed prosthetic heart valve is determined to be in the desired position relative to the native annulus (e.g. via visualization under fluoroscopy), a fluid (typically a liquid although gas could be used as well) such as saline is pushed via a syringe (manually, automatically, or semi-automatically) through the balloon catheter to cause the balloon to begin to fill and expand, and thus cause the overlying prosthetic heart valve to expand into the native annulus.

Prosthetic heart valves (including TAVR) may experience high stress in certain areas, especially near the leaflet commissure attachment to the stent frame. These high stress areas may increase the risk of valve failure, reduce the longevity of the implant or lead to suboptimal function due to the stiffness mismatch between stent and valve components (e.g., leaflets).

### BRIEF SUMMARY OF THE DISCLOSURE

In some embodiments, a prosthetic heart valve includes a collapsible and expandable stent having an inflow end and an outflow end, and a plurality of struts defining a plurality of cells, the stent being formed of a first material, a plurality of commissure attachment features formed of a second material that is different than the first material of the stent, and a collapsible and expandable valve assembly including a plurality of leaflets connected to the plurality of commissure attachment features.

In some embodiments, a prosthetic heart valve includes a collapsible and expandable stent having an inflow end and an outflow end, and a plurality of struts defining a plurality of cells, the stent having a first thickness a plurality of commissure attachment features having a second thickness that is less than the first thickness of the stent, and a collapsible and expandable valve assembly including a plurality of leaflets connected to the plurality of commissure attachment features.

In some embodiments, a method of forming a prosthetic heart valve includes providing a collapsible and expandable stent having an inflow end and an outflow end, and a plurality of struts defining a plurality of cells, forming a plurality of commissure attachment features, the plurality of commissure attachment features being more flexible than the collapsible and expandable stent, and coupling a collapsible and expandable valve assembly including a plurality of leaflets connected to the plurality of commissure attachment features.

### BRIEF DESCRIPTION OF THE DRAWINGS

FIG. 1A is a perspective view of a stent of a prosthetic heart valve according to an embodiment of the disclosure.

FIG. 1B is a schematic front view of a section of the stent of FIG. 1A.

FIG. 1C is a schematic front view of a section of a stent according to an alternate embodiment of the prosthetic heart valve of FIG. 1A.

FIGS. 1D-E are front views of the stent section of FIG. 1C in a collapsed and expanded state, respectively.

FIGS. 1F-G are side views of a portion of the stent according to the embodiment of FIG. 1C in a collapsed and expanded state, respectively.

FIG. 1H is a flattened view of the stent according to the embodiment of FIG. 1C, as if cut and rolled flat.

FIGS. 1I-J are front and side views, respectively, of a prosthetic heart valve including the stent of FIG. 1C.

FIG. 1K illustrates the view of FIG. 1H with an additional outer cuff provided on the stent.

FIG. 2A illustrates a prosthetic heart valve crimped over a balloon of a delivery device.

FIG. 2B is a schematic view of the balloon of FIG. 2A after having been inflated.

FIG. 3 is an illustration of four finite element models of certain valves and the visualized stress concentrations on the leaflets.

FIGS. 4A-D are schematic illustrations showing a stent with a commissure feature, and alternative methods of forming the stent to increase flexibility.

FIGS. 5A-D are schematic illustrations of a stent and potential deflection of the commissure features based on certain modifications.

FIG. 6 is a diagram showing the effects of cold-working a material on ductility, hardness and strength, and the potential application to commissure features.

FIGS. 7A-B are schematic illustrations of a stent having deflectable sections at the inflow and outflow ends, respectively.

### DETAILED DESCRIPTION

As used herein, the term “inflow end” when used in connection with a prosthetic heart valve refers to the end of the prosthetic valve into which blood first enters when the prosthetic valve is implanted in an intended position and orientation, while the term “outflow end” refers to the end of the prosthetic valve where blood exits when the prosthetic valve is implanted in the intended position and orientation. Thus, for a prosthetic aortic valve, the inflow end is the end nearer the left ventricle while the outflow end is the end nearer the aorta. The intended position and orientation are used for the convenience of describing the valve disclosed herein, however, it should be noted that the use of the valve is not limited to the intended position and orientation but may be deployed in any type of lumen or passageway. For example, although the prosthetic heart valve is described herein as a prosthetic aortic valve, the same or similar structures and features can be employed in other heart valves, such as the pulmonary valve, the mitral valve, or the tricuspid valve. Further, the term “proximal,” when used in connection with a delivery device or system, refers to a direction relatively close to the user of that device or system when being used as intended, while the term “distal” refers to a direction relatively far from the user of the device. In other words, the leading end of a delivery device or system is positioned distal to the trailing end of the delivery device or system, when being used as intended. As used herein, the terms “substantially,” “generally,” “approximately,” and “about” are intended to mean that slight deviations from absolute are included within the scope of the term so modified. As used herein, the stent may assume an “expanded state” and a “collapsed state,” which refer to the relative radial size of the stent.

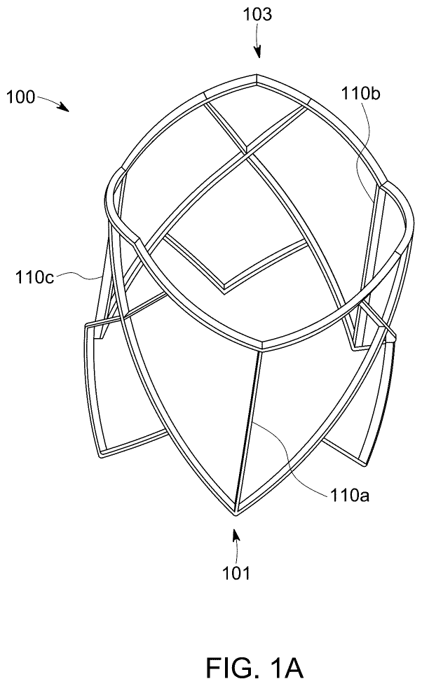

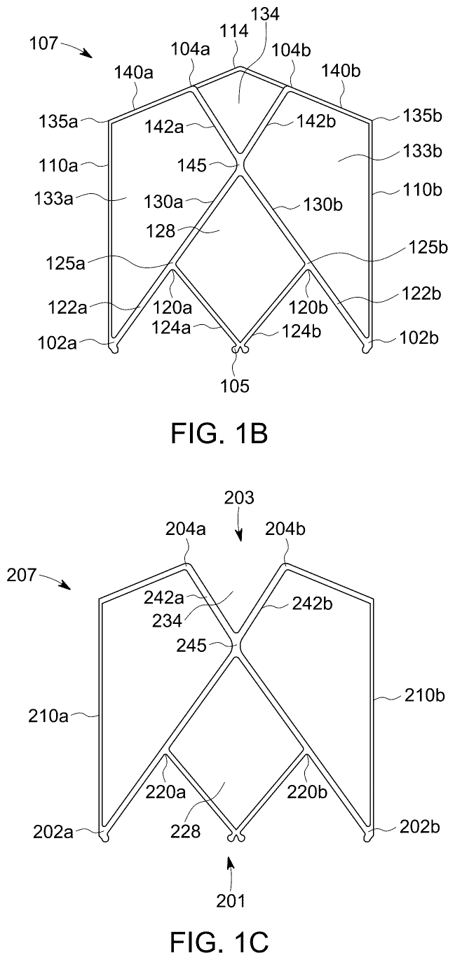

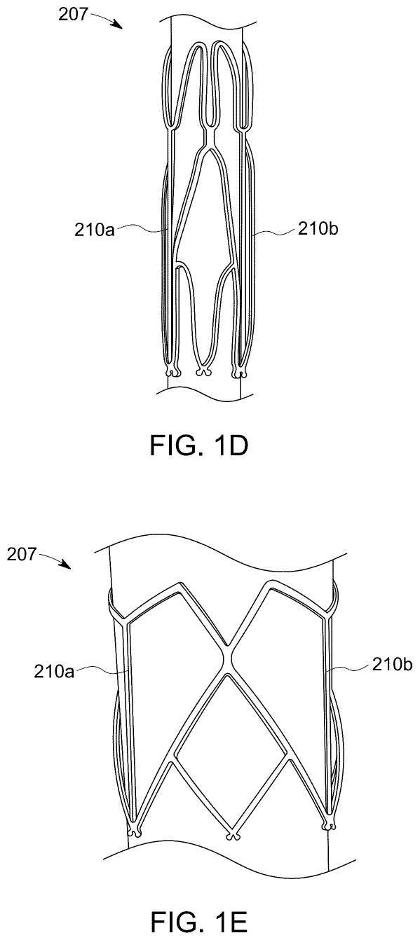

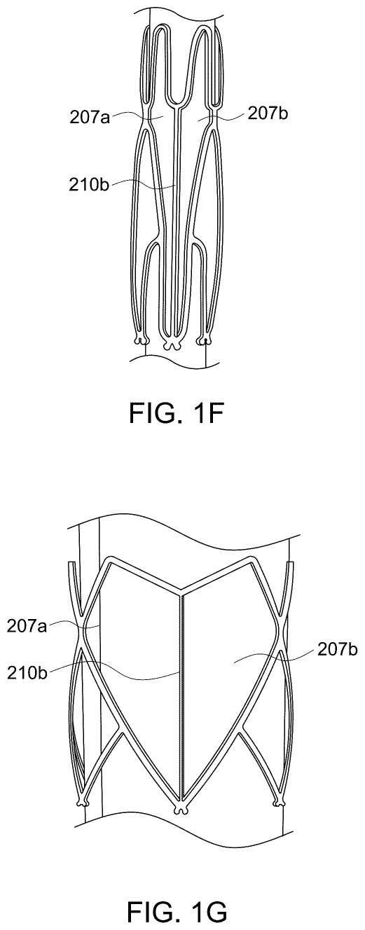

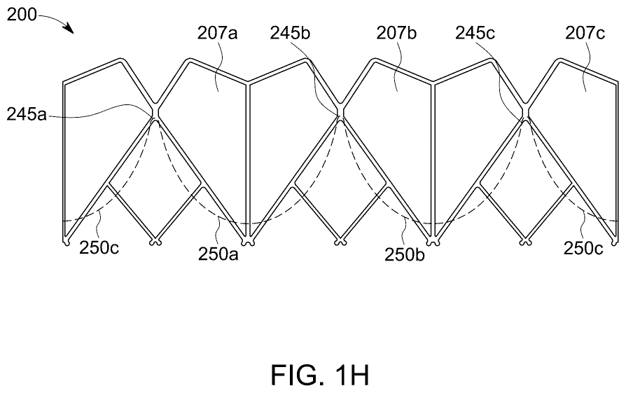

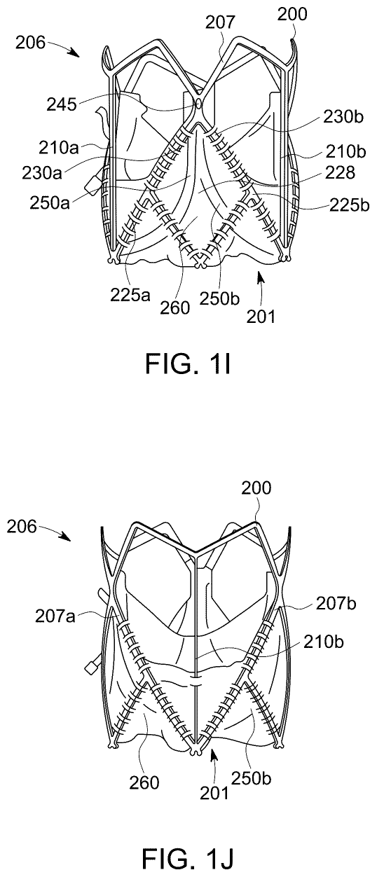

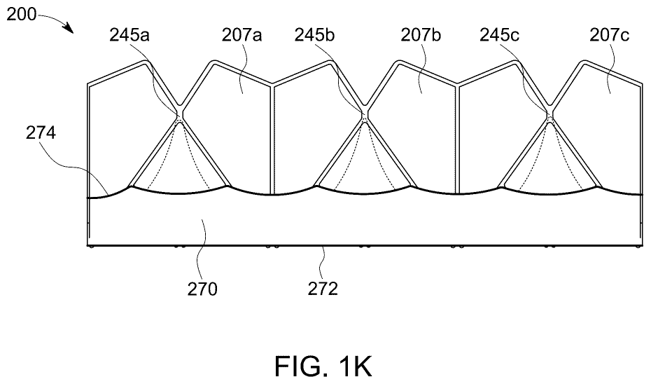

FIG. 1A illustrates a perspective view of a stent 100 of a prosthetic heart valve according to an embodiment of the disclosure. Stent 100 may include a frame extending in an axial direction between an inflow end 101 and an outflow end 103. Stent 100 includes three generally symmetric sections, wherein each section spans about 120 degrees around the circumference of stent 100. Stent 100 includes three vertical struts 110a, 110b, 110c, that extend in an axial direction substantially parallel to the direction of blood flow through the stent, which may also be referred to as a central longitudinal axis. Each vertical strut 110a, 110b, 110c may extend substantially the entire axial length between the inflow end 101 and the outflow end 103 of the stent 100 and may be disposed between and shared by two sections. In other words, each section is defined by the portion of stent 100 between two vertical struts. Thus, each vertical strut 110a, 110b, 110c is also separated by about 120 degrees around the circumference of stent 100. It should be understood that, if stent 100 is used in a prosthetic heart valve having three leaflets, the stent may include three sections as illustrated. However, in other embodiments, if the prosthetic heart valve has two leaflets, the stent may only include two of the sections.

FIG. 1B illustrates a schematic view of a stent section 107 of stent 100, which will be described herein in greater detail, and which is representative of all three sections. Stent section 107 depicted in FIG. 1B includes a first vertical strut 110a and a second vertical strut 110b. First vertical strut 110a extends axially between a first inflow node 102a and a first outer node 135a. Second vertical strut 110b extends axially between a second inflow node 102b and a second outer node 135b. As is illustrated, the vertical struts 110a, 110b may extend almost the entire axial length of stent 100. In some embodiments, stent 100 may be formed as an integral unit, for example by laser cutting the stent from a tube. The term “node” may refer to where two or more struts of the stent 100 meet one another. A pair of sequential inverted V's extends between inflow nodes 102a, 102b, which includes a first inflow inverted V 120a and a second inflow inverted V 120b coupled to each other at an inflow node 105. First inflow inverted V 120a comprises a first outer lower strut 122a extending between first inflow node 102a and a first central node 125a. First inflow inverted V 120a further comprises a first inner lower strut 124a extending between first central node 125a and inflow node 105. A second inflow inverted V 120b comprises a second inner lower strut 124b extending between inflow node 105 and a second central node 125b. Second inflow inverted V 120b further comprises a second outer lower strut 122b extending between second central node 125b and second inflow node 102b. Although described as inverted V's, these structures may also be described as half-cells, each half cell being a half-diamond cell with the open portion of the half-cell at the inflow end 101 of the stent 100.

Stent section 107 further includes a first central strut 130a extending between first central node 125a and an upper node 145. Stent section 107 also includes a second central strut 130b extending between second central node 125b and upper node 145. First central strut 130a, second central strut 130b, first inner lower strut 124a and second inner lower strut 124b form a diamond cell 128. Stent section 107 includes a first outer upper strut 140a extending between first outer node 135 and a first outflow node 104a. Stent section 107 further includes a second outer upper strut 140b extending between second outer node 135b and a second outflow node 104b. Stent section 107 includes a first inner upper strut 142a extending between first outflow node 104a and upper node 145. Stent section 107 further includes a second inner upper strut 142b extending between upper node 145 and second outflow node 104b. Stent section 107 includes an outflow inverted V 114 which extends between first and second outflow nodes 104a, 104b. First vertical strut 110a, first outer upper strut 140a, first inner upper strut 142a, first central strut 130a and first outer lower strut 122a form a first generally kite-shaped cell 133a. Second vertical strut 110b, second outer upper strut 140b, second inner upper strut 142b, second central strut 130b and second outer lower strut 122b form a second generally kite-shaped cell 133b. First and second kite-shaped cells 133a, 133b are symmetric and opposite each other on stent section 107. Although the term “kite-shaped,” is used above, it should be understood that such a shape is not limited to the exact geometric definition of kite-shaped. Outflow inverted V 114, first inner upper strut 142a and second inner upper strut 142b form upper cell 134. Upper cell 134 is generally kite-shaped and axially aligned with diamond cell 128 on stent section 107. It should be understood that, although designated as separate struts, the various struts described herein may be part of a single unitary structure as noted above. However, in other embodiments, stent 100 need not be formed as an integral structure and thus the struts may be different structures (or parts of different structures) that are coupled together.

FIG. 1C illustrates a schematic view of a stent section 207 according to an alternate embodiment of the disclosure. Unless otherwise stated, like reference numerals refer to like elements of above-described stent 100 but within the 200-series of numbers. Stent section 207 is substantially similar to stent section 107, including inflow nodes 202a, 202b, vertical struts 210a, 210b, first and second inflow inverted V's 220a, 220b and outflow nodes 204a, 204b. The structure of stent section 207 departs from that of stent section 107 in that it does not include an outflow inverted V. The purpose of an embodiment having such structure of stent section 207 shown in FIG. 1C is to reduce the required force to expand the outflow end 203 of the stent 200, compared to stent 100, to promote uniform expansion relative to the inflow end 201. Outflow nodes 204a, 204b are connected by a properly oriented V formed by first inner upper strut 242a, upper node 245 and second inner upper strut 242b. In other words, struts 242a, 242b may form a half diamond cell 234, with the open end of the half-cell oriented toward the outflow end 203. Half diamond cell 234 is axially aligned with diamond cell 228. Adding an outflow inverted V coupled between outflow nodes 204a, 204b contributes additional material that increases resistance to modifying the stent shape and requires additional force to expand the stent. The exclusion of material from outflow end 203 decreases resistance to expansion on outflow end 203, which may promote uniform expansion of inflow end 201 and outflow end 203. In other words, the inflow end 201 of stent 200 does not include continuous circumferential structure, but rather has mostly or entirely open half-cells with the open portion of the half-cells oriented toward the inflow end 201, whereas most of the outflow end 203 includes substantially continuous circumferential structure, via struts that correspond with struts 140a, 140b. All else being equal, a substantially continuous circumferential structure may require more force to expand compared to a similar but open structure. Thus, the inflow end 101 of stent 100 may require more force to radially expand compared to the outflow end 103. By omitting inverted V 114, resulting in stent 200, the force required to expand the outflow end 203 of stent 200 may be reduced to an amount closer to the inflow end 201.

FIG. 1D shows a front view of stent section 207 in a collapsed state and FIG. 1E shows a front view of stent section 207 in an expanded state. It should be understood that stent 200 in FIGS. 1D-E is illustrated with an opaque tube extending through the interior of the stent, purely for the purpose of helping illustrate the stent, and which may represent a balloon over which the stent section 207 is crimped. As described above, a stent comprises three symmetric sections, each section spanning about 120 degrees around the circumference of the stent. Stent section 207 illustrated in FIGS. 1D-E is defined by the region between vertical struts 210a, 210b. Stent section 207 is representative of all three sections of the stent. Stent section 207 has an arcuate structure such that when three sections are connected, they form one complete cylindrical shape. FIGS. 1F-G illustrate a portion of the stent from a side view. In other words, the view of stent 200 in FIGS. 1F-G is rotated about 60 degrees compared to the view of FIGS. 1D-E. The view of the stent depicted in FIGS. 1F-G is centered on vertical strut 210b showing approximately half of each of two adjacent stent sections 207a, 207b on each side of vertical strut 210b. Sections 207a, 207b surrounding vertical strut 210b are mirror images of each other. FIG. 1F shows stent sections 207a, 207b in a collapsed state whereas FIG. 1G shows stent sections 207a, 207b in an expanded state.

FIG. 1H illustrates a flattened view of stent 200 including three stent sections 207a, 207b, 207c, as if the stent has been cut longitudinally and laid flat on a table. As depicted, sections 207a, 207b, 207c are symmetric to each other and adjacent sections share a common vertical strut. As described above, stent 200 is shown in a flattened view, but each section 207a, 207b, 207c has an arcuate shape spanning 120 degrees to form a full cylinder. Further depicted in FIG. 1H are leaflets 250a, 250b, 250c coupled to stent 200. However, it should be understood that only the connection of leaflets 250a-c is illustrated in FIG. 1H. In other words, each leaflet 250a-c would typically include a free edge, with the free edges acting to coapt with one another to prevent retrograde flow of blood through the stent 200, and the free edges moving radially outward toward the interior surface of the stent to allow antegrade flow of blood through the stent. Those free edges are not illustrated in FIG. 1H. Rather, the attached edges of the leaflets 250a-c are illustrated in dashed lines in FIG. 1H. Although the attachment may be via any suitable modality, the attached edges may be preferably sutured to the stent 200 and/or to an intervening cuff or skirt between the stent and the leaflets 250a-c. Each of the three leaflets 250a, 250b, 250c, extends about 120 degrees around stent 200 from end to end and each leaflet includes a belly that may extend toward the radial center of stent 200 when the leaflets are coapted together. Each leaflet extends between the upper nodes of adjacent sections. First leaflet 250a extends from first upper node 245a of first stent section 207a to second upper node 245b of second stent section 207b. Second leaflet 250b extends from second upper node 245b to third upper node 245c of third stent section 207c. Third leaflet 250c extends from third upper node 245c to first upper node 245a. As such, each upper node includes a first end of a first leaflet and a second end of a second leaflet coupled thereto. In the illustrated embodiment, each end of each leaflet is coupled to its respective node by suture. However, any coupling means may be used to attach the leaflets to the stent. It is further contemplated that the stent may include any number of sections and/or leaflets. For example, the stent may include two sections, wherein each section extends 180 degrees around the circumference of the stent. Further, the stent may include two leaflets to mimic a bicuspid valve. Further, it should be noted that each leaflet may include tabs or other structures (not illustrated) at the junction between the free edges and attached edges of the leaflets, and each tab of each leaflet may be coupled to a tab of an adjacent leaflet to form commissures. In the illustrated embodiment, the leaflet commissures are illustrated attached to nodes where struts intersect. However, in other embodiments, the stent 200 may include commissure attachment features built into the stent to facilitate such attachment. For example, commissures attachment features may be formed into the stent 200 at nodes 245a-c, with the commissure attachment features including one or more apertures to facilitate suturing the leaflet commissures to the stent. Further, leaflets 250a-c may be formed of a biological material, such as animal pericardium, or may otherwise be formed of synthetic materials, such as plastics, fabrics, and/or polymers, including ultra-high molecular weight polyethylene (UHMWPE).

FIGS. 1I-J illustrate prosthetic heart valve 206, which includes stent 200, a cuff 260 coupled to stent 200 (for example via sutures) and leaflets 250a, 250b, 250c attached to stent 200 and/or cuff 260 (for example via sutures). Prosthetic heart valve 206 is intended for use in replacing an aortic valve, although the same or similar structures may be used in a prosthetic valve for replacing other heart valves. Cuff 260 is disposed on a luminal or interior surface of stent 200, although the cuff could be disposed alternately or additionally on an abluminal or exterior surface of the stent. The cuff 260 may include an inflow end disposed substantially along inflow end 201 of stent 200. FIG. 1I shows a front view of valve 206 showing one stent portion 207 between vertical struts 210a, 210b including cuff 260 and an outline of two leaflets 250a, 250b sutured to cuff 260. Different methods of suturing leaflets to the cuff as well as the leaflets and/or cuff to the stent may be used, many of which are described in U.S. Pat. No. 9,326,856 which is hereby incorporated by reference. In the illustrated embodiment, the upper (or outflow) edge of cuff 260 is sutured to first central node 225a, upper node 245 and second central node 225b, extending along first central strut 230a and second central strut 230b. The upper (or outflow) edge of cuff 260 continues extending approximately between the second central node of one section and the first central node of an adjacent section. Cuff 260 extends between upper node 245 and inflow end 201. Thus, cuff 260 covers the cells of stent portion 207 formed by the struts between upper node 245 and inflow end 201, including diamond cell 228. FIG. 1J illustrates a side view of stent 200 including cuff 260 and an outline of leaflet 250b. In other words, the view of valve 206 in FIG. 1J is rotated about 60 degrees compared to the view of FIG. 1I. The view depicted in FIG. 1J is centered on vertical strut 210b showing approximately half of each of two adjacent stent sections 207a, 207b on each side of vertical strut 210b. Sections 207a, 207b surrounding vertical strut 210b are mirror images of each other. As described above, the cuff may be disposed on the stent's interior or luminal surface, its exterior or abluminal surface, and/or on both surfaces. A cuff ensures that blood does not just flow around the valve leaflets if the valve or valve assembly are not optimally seated in a valve annulus. A cuff, or a portion of a cuff disposed on the exterior of the stent, can help retard leakage around the outside of the valve (the latter known as paravalvular leakage or “PV” leakage). In the embodiment illustrated in FIGS. 1I-J, the cuff 260 only covers about half of the stent 200, leaving about half of the stent uncovered by the cuff. With this configuration, less cuff material is required compared to a cuff that covers more or all of the stent 200. Less cuff material may allow for the prosthetic heart valve 206 to crimp down to a smaller profile when collapsed. It is contemplated that the cuff may cover any amount of surface area of the cylinder formed by the stent. For example, the upper edge of the cuff may extend straight around the circumference of any cross section of the cylinder formed by the stent. Cuff 260 may be formed of any suitable material, including a biological material such as animal pericardium, or a synthetic material such as UHMWPE.

As noted above, FIGS. 1I-J illustrate a cuff 260 positioned on an interior of the stent 200. An example of an additional outer cuff 270 is illustrated in FIG. 1K. It should be understood that outer cuff 270 may take other shapes than that shown in FIG. 1K. The outer cuff 270 shown in FIG. 1K may be included without an inner cuff 260, but preferably is provided in addition to an inner cuff 260. The outer cuff 270 may be formed integrally with the inner cuff 260 and folded over (e.g. wrapped around) the inflow edge of the stent, or may be provided as a member that is separate from inner cuff 260. Outer cuff 270 may be formed of any of the materials described herein in connection with inner cuff 260. In the illustrated embodiment, outer cuff 270 includes an inflow edge 272 and an outflow edge 274. If the inner cuff 260 and outer cuff 270 are formed separately, the inflow edge 272 may be coupled to an inflow end of the stent 200 and/or an inflow edge of the inner cuff 260, for example via suturing, ultrasonic welding, or any other suitable attachment modality. The coupling between the inflow edge 272 of the outer cuff 270 and the stent 200 and/or inner cuff 260 preferably results in a seal between the inner cuff 260 and outer cuff 270 at the inflow end of the prosthetic heart valve so that any retrograde blood that flows into the space between the inner cuff 260 and outer cuff 270 is unable to pass beyond the inflow edges of the inner cuff 260 and outer cuff 270. The outflow edge 274 may be coupled at selected locations around the circumference of the stent 200 to struts of the stent 200 and/or to the inner cuff 260, for example via sutures. With this configuration, an opening may be formed between the inner cuff 260 and outer cuff 270 circumferentially between adjacent connection points, so that retrograde blood flow will tend to flow into the space between the inner cuff 260 and outer cuff 270 via the openings, without being able to continue passing beyond the inflow edges of the cuffs. As blood flows into the space between the inner cuff 260 and outer cuff 270, the outer cuff 270 may billow outwardly, creating even better sealing between the outer cuff 270 and the native valve annulus against which the outer cuff 270 presses. The outer cuff 270 may be provided as a continuous cylindrical member, or a strip that is wrapped around the outer circumference of the stent 200, with side edges, which may be parallel or non-parallel to a center longitudinal axis of the prosthetic heart valve, attached to each other so that the outer cuff 270 wraps around the entire circumference of the stent 200.

The stent may be formed from biocompatible materials, including metals and metal alloys such as cobalt chrome (or cobalt chromium) or stainless steel, although in some embodiments the stent may be formed of a shape memory material such as nitinol or the like. The stent is thus configured to collapse upon being crimped to a smaller diameter and/or expand upon being forced open, for example via a balloon within the stent expanding, and the stent will substantially maintain the shape to which it is modified when at rest. The stent may be crimped to collapse in a radial direction and lengthen (to some degree) in the axial direction, reducing its profile at any given cross-section. The stent may also be expanded in the radial direction and foreshortened (to some degree) in the axial direction.

The prosthetic heart valve may be delivered via any suitable transvascular route, for example including transapically or transfemorally. Generally, transapical delivery utilizes a relatively stiff catheter that pierces the apex of the left ventricle through the chest of the patient, inflicting a relatively higher degree of trauma compared to transfemoral delivery. In a transfemoral delivery, a delivery device housing the valve is inserted through the femoral artery and threaded against the flow of blood to the left ventricle. In either method of delivery, the valve may first be collapsed over an expandable balloon while the expandable balloon is deflated. The balloon may be coupled to or disposed within a delivery system, which may transport the valve through the body and heart to reach the aortic valve, with the valve being disposed over the balloon (and, in some circumstance, under an overlying sheath). Upon arrival at or adjacent the aortic valve, a surgeon or operator of the delivery system may align the prosthetic valve as desired within the native valve annulus while the prosthetic valve is collapsed over the balloon. When the desired alignment is achieved, the overlying sheath, if included, may be withdrawn (or advanced) to uncover the prosthetic valve, and the balloon may then be expanded causing the prosthetic valve to expand in the radial direction, with at least a portion of the prosthetic valve foreshortening in the axial direction.

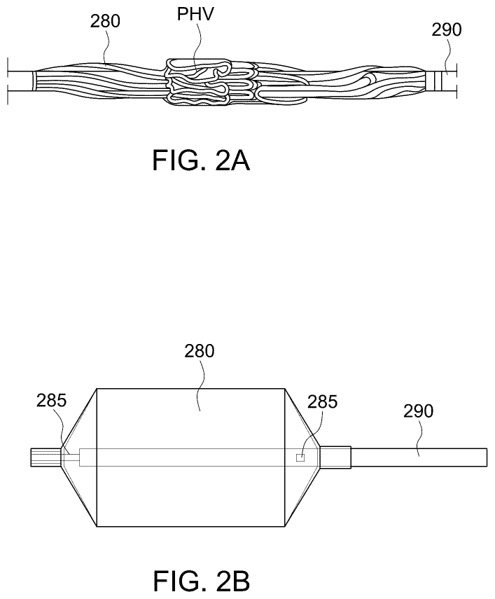

Referring to FIG. 2A, an example of a prosthetic heart valve PHV, which may include a stent similar to stents 100 or 200, is shown crimped over a balloon 280 of a balloon catheter 290 while the balloon 280 is in a deflated condition. It should be understood that other components of the delivery device, such as a handle used for steering and/or deployment, as well as a syringe for inflating the balloon 280, are omitted from FIGS. 2A-B. The prosthetic heart valve PHV may be delivered intravascularly, for example through the femoral artery, around the aortic arch, and into the native aortic valve annulus, while in the crimped condition shown in FIG. 2A. Once the desired position is obtained, fluid may be pushed through the balloon catheter 290 to inflate the balloon 280, as shown in FIG. 2B. FIG. 2B omits the prosthetic heart valve PHV, but it should be understood that, as the balloon 280 inflates, it forces the prosthetic heart valve PHV to expand into the native aortic valve annulus (although it should be understood that other heart valves may be replaced using the concepts described herein). In the illustrated example, fluid flows from a syringe (not shown) into the balloon 280 through a lumen within balloon catheter 290 and into one or more ports 285 located internal to the balloon 280. In the particular illustrated example of FIG. 2B, a first port 285 may be one or more apertures in a side wall of the balloon catheter 290, and a second port 285 may be the distal open end of the balloon catheter 290, which may terminate within the interior space of the balloon 280.

During normal operation of a prosthetic heart valve, the prosthetic leaflets open and close cyclically as the chambers of the heart contract and relax. For example, when the left ventricle relaxes and the left atrium contracts, the mitral valve opens and the aortic valve closes. For a prosthetic aortic valve, as the left ventricle relaxes, the prosthetic leaflets coapt to prevent blood from flowing in the retrograde direction from the aorta back into the left ventricle. As the prosthetic leaflets open and close, and particularly when they close, the prosthetic leaflets can encounter stress as the prosthetic leaflets resist the pressure gradient across the closed valve assembly. This stress may largely act at the point(s) where the prosthetic leaflets are affixed to the frame (or an intermediary component).

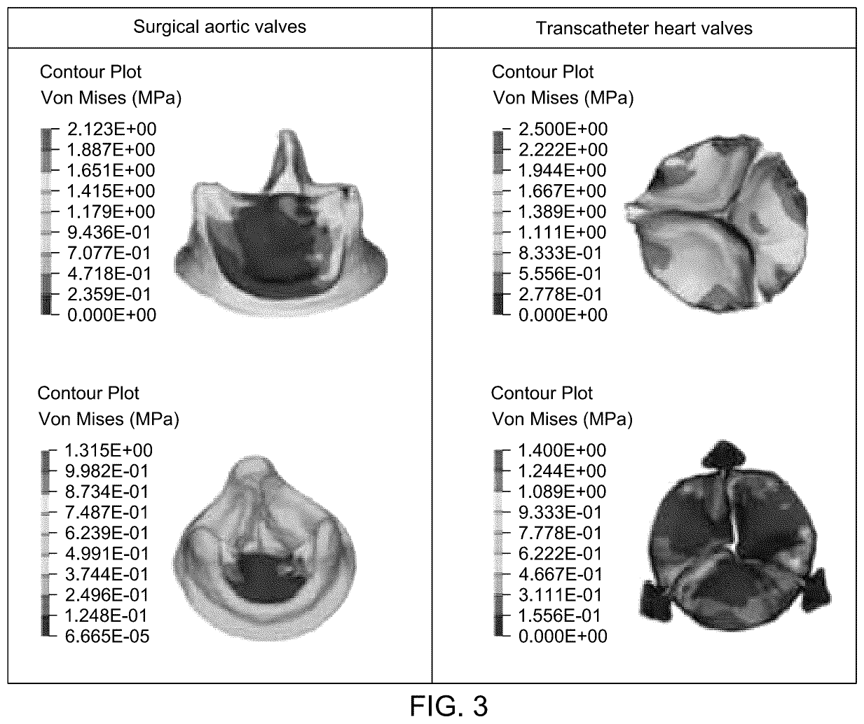

Because prosthetic heart valves may need to last years, decades, or more, it may be important to minimize the amount of stress experienced by the prosthetic leaflets during normal operation to reduce the amount of wear and tear on the prosthetic leaflets, since such wear and tear may reduce the longevity of the prosthetic leaflets. One way to reduce stress on the prosthetic leaflets is to allow for deflection of the structure(s) to which the prosthetic leaflets are attached. For example, if prosthetic leaflets are directly sutured to a commissure attachment feature of a frame, allowing the frame to deflect slightly (e.g., about 1 mm) as the prosthetic leaflets close may help reduce the stresses on the prosthetic leaflets as they coapt. FIG. 3 is an illustration showing four finite element models of certain common surgical and transcatheter heart valves and the visualized stress concentrations on the leaflets. As shown, the highest stress concentrations in each figure are found adjacent the commissure attachment features. Additionally, because stent materials (e.g., metals or polymers) are generally stiffer than leaflet materials (e.g., tissue or thin film synthetics), it would be desirable for the stent to have some flexibility in the commissure region. This can be referred to as stent commissure deflection. The present disclosure describes several methods and configurations to increase stent commissure deflection in relatively stiff materials, such as stainless steel or cobalt chromium.

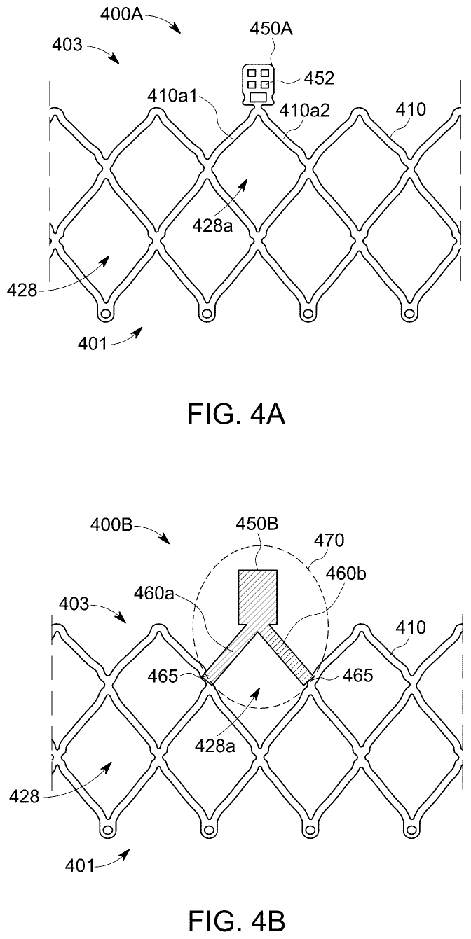

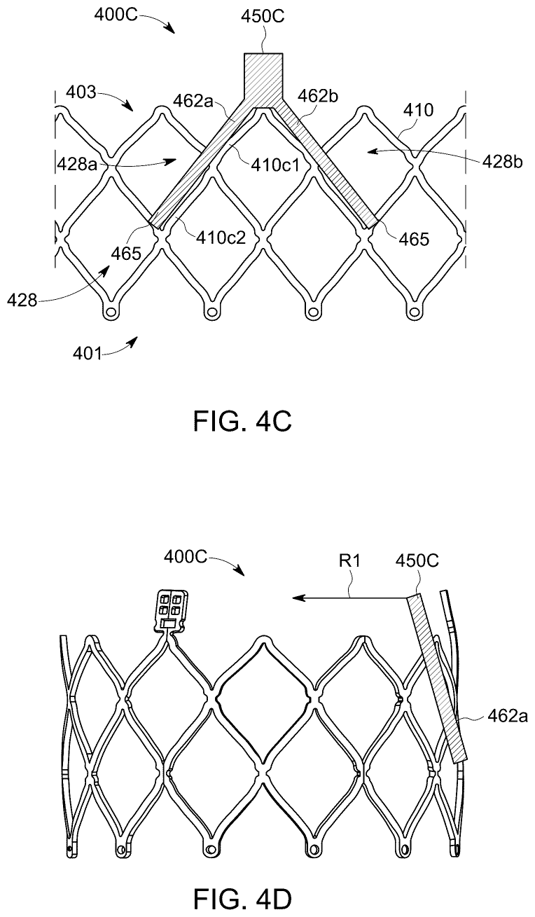

FIGS. 4A-D illustrate several variations of a stent of a prosthetic heart valve according to a first embodiment of the disclosure. In FIG. 4A, stent 400A may include a frame extending in an axial direction between an inflow end 401 and an outflow end 403. In this example, stent 400A includes angled struts 410 that collectively form diamond-shaped cells 428. Two full rows of diamond-shaped cells 428 are shown in FIG. 4A, although it will be understood that a stent may be formed of a single row of cells, two rows of cells, or three or more rows of cells. In this example, each of the two rows includes cells 428 that are substantially diamond-shaped, although other shapes are also possible. It will be understood that the size and/or shape of each cell may vary between rows, or that the size and/or shape of cells may vary within a given row of cells. In this example, a commissure attachment feature 450A is coupled to an apex of cell 428a, the apex being formed by two struts 4101,410a2 of a cell 428a in the upper row. Commissure attachment feature 450A may be substantially rectangular and include a plurality of eyelets 452 of different shapes and sizes. Other variations of the commissure attachment feature shape and connection to a stent are shown in U.S. Pat. No. 9,693,861, which is hereby incorporated by reference in its entirety as if fully set forth herein, and any of the commissure attachment features described therein are contemplated as being possible configurations to be combined with the present disclosure. It should be understood that, although designated as separate struts, the various struts described herein may be part of a single unitary structure as noted above. However, in other embodiments, stent 400A need not be formed as an integral structure and thus the struts may be different structures (or parts of different structures) that are coupled together.

In one variation, shown in FIG. 4B, stent 400B includes a frame extending in an axial direction between an inflow end 401 and an outflow end 403 that includes struts 410 that collectively form cells 428. Stent 400B is substantially similar to stent 400A except in the configuration of the commissure attachment feature 450B. In this example, stent 400B includes two flexible connecting struts 460a,460b that replace two struts of cell 428a, and couple the commissure attachment feature 450B to stent 400B. Flexible connecting struts 460a,460b may comprise a different material than the remaining struts 410 of stent 400B. For example, flexible connecting struts 460a,460b may comprise a secondary material (e.g., an alloy such as nitinol or titanium, a polymer such as acetal or PTFE, or other) that is more flexible than the primary material of the rest of the stent 400B (e.g., stainless steel or cobalt chromium). It will be understood that the commissure attachment feature 450B itself may also be formed of the secondary material, and may be more flexible than the remainder of stent 400B to create a flexible zone 470. In this example, flexible connecting struts 460a,460b may be attached to other struts of the stent at junctions 465 via welding, bonding, suturing, or by being captured between other members of the TAVI valve such as an inner and outer cuff for instance. For example, instead of members of two stent materials being attached directly to each other, they may be attached indirectly by placing an inner and outer cuff and/or fabric material therebetween and suturing those members together to capture the flexible material, for instance. In this example, flexible connecting struts 460a,460b are attached to two side apices or intersections of struts at junctions 465, but it will be understood that flexible connecting struts 460a,460b may also be attached to a single strut (i.e., not at the apices of a cell, nor at the intersection of two struts).

In a second variation, shown in FIG. 4C, stent 400C includes a frame extending in an axial direction between an inflow end 401 and an outflow end 403 that includes struts 410 that collectively form cells 428. Stent 400C is substantially similar to stents 400A,400B except in the configuration of the commissure attachment feature. In this example, stent 400C includes two flexible connecting struts 462a,462b that attach a commissure attachment feature 450C to stent 400C. Flexible connecting struts 462a,462b and/or commissure attachment feature 450C may be formed of a different material than the remainder of stent 400C as previously described. In this example, flexible connecting struts 462a,462b do not replace struts of the stent, but additionally couple adjacent the lower apices of cells 428a,428b at junctions 465. Flexible connecting struts 462a,462b may be coupled directly to the intersection of two struts, or close to the intersection but only to a single strut near the intersection. Additionally, flexible connecting struts 462a,462b may be longer than flexible connecting struts 460a,460b and span an entirety of a cell or most of a cell. In some examples, flexible connecting struts 462a,462b span half of the length of stent 400C or more than half the length of a stent (e.g., each flexible connecting strut may be equal to or longer than the combined lengths of struts 410c1,410c2). FIG. 4D illustrates stent 400C and specifically shows that commissure attachment feature 450C, coupled to flexible connecting struts 462a,462b may be more flexible and allow the commissure attachment feature 450C to more easily radially deflect, or to deflect to a greater degree, toward the center of the stent 400C in the direction of arrow R1.

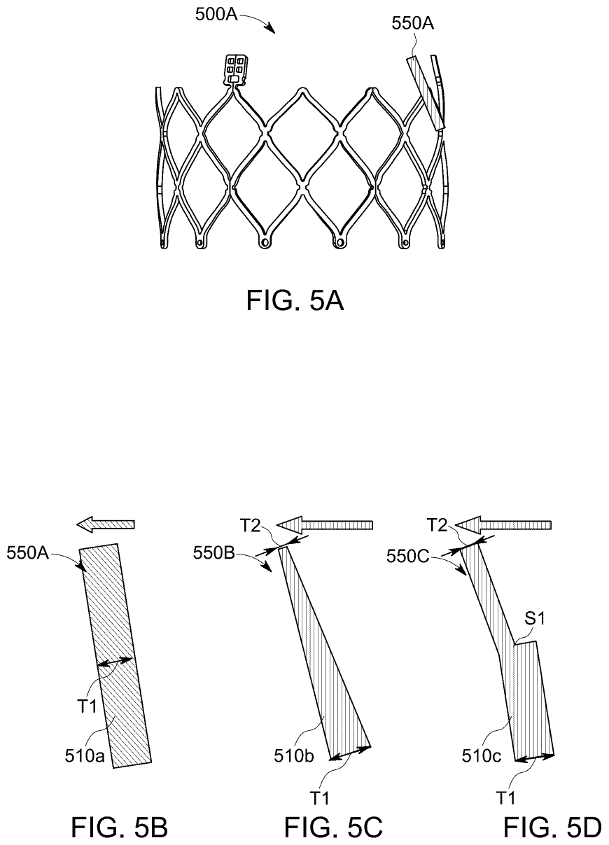

FIG. 5A-D illustrate the effects of modifying certain cross-sectional areas of features of a stent. In FIG. 5A, a stent 500A includes a commissure attachment feature 550A that is being deflected toward the center of the stent. As shown in FIGS. 5B-D, the cross-sectional areas of the commissure attachment features 550A-C and/or the struts that connect them to the stent may be modified. In FIG. 5B, a commissure attachment feature 550A and a connecting strut 510a may have a constant cross-sectional area from one end to the other. This is common when a stent is formed by laser cutting a tube having a constant wall thickness. In this example, a first wall thickness T1 of the commissure attachment feature 550A is the same as the thickness of the connecting strut 510a (i.e., there is a uniform thickness between the commissure attachment feature and the connecting struts). However, this first wall thickness T1 may be smaller than the wall thickness of the rest of the stent.

In a first variation, shown in FIG. 5C, instead of, or in addition to, being thinner than the rest of the stent, commissure attachment feature 550C may be thinner than connecting strut 510b. In this example, commissure attachment feature 550C has a second wall thickness T2 and connecting strut 510b has a first wall thickness T1, the first wall thickness T1 being greater than the second wall thickness T2. In some examples, the thickness of the two members gradually or linear decreases from the connecting strut 510b to the tip of commissure attachment feature 550C. In some examples, the ratio of the first wall thickness T1 to the second wall thickness T2 is 1.5:1, 2:1, or 3:1, and this reduction from one thickness to the other may be gradual, linear, or non-linear.

In a second variation, shown in FIG. 5D, commissure attachment feature 550D is thinner than connecting strut 510c. In this example, commissure attachment feature 550D has a thickness T2 and connecting strut 510c has a thickness T1, the first wall thickness T1 being greater than the second wall thickness T2. In this example, the second wall thickness T2 of commissure attachment feature 550D is constant and the first wall thickness T1 of connecting strut 510c is also constant, but a transitioning step S1 is formed between the two members. In some examples, the ratio of the first wall thickness T1 to the second wall thickness T2 is 1.5:1, 2:1, or 3:1. Here again, it will be understood that the first wall thickness T1 may be equal to or less than the rest of the thickness of the stent.

In some examples, a stent may be formed of a uniform thickness and the modifications to reduce the cross-sectional area or thickness of certain components may be completed after the stent is cut. For example, the reduction in thickness may be achieved by machining, targeted grit blasting or electropolishing the commissure attachment features and/or the connecting struts. Alternatively, a different form of the native metal such as wire may be used to form the thinned regions of the commissure attachment features and/or the connecting struts instead of laser cut tubing.

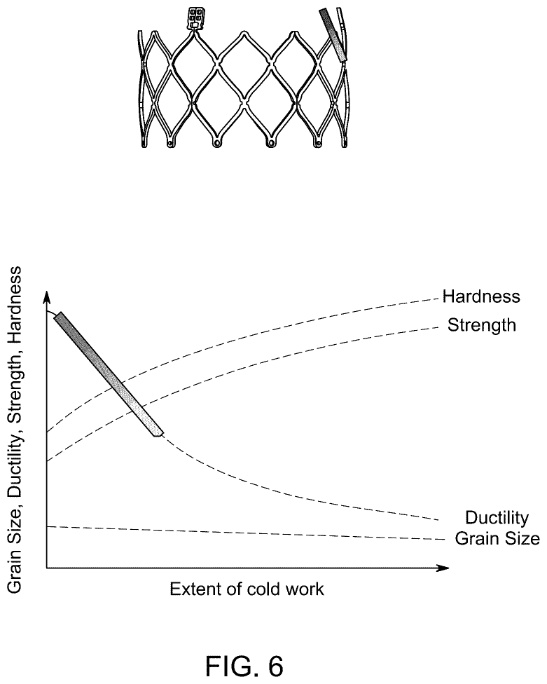

In yet another embodiment, flexibility at, or near, the commissure attachment feature may be increased by modifying the microstructure of certain portions of the stent at or near the commissure region. FIG. 6 illustrates certain effects of a cold working process on grain size, ductility, strength and hardness of a metal. Additional details of the effects of the cold working process may be found in Bhaduri, A. (2018). Mechanical Properties and Working of Metals and Alloys. Germany: Springer Singapore, which is hereby incorporated by reference in its entirety as if fully set forth herein. In some examples, the commissure attachment features and/or the connecting struts may undergo a treatment to modify its physical parameter(s) and create a modified microstructure. In some examples, the commissure attachment features, the connecting struts, the stent or any combination of these may undergo an annealing process or heat treatment. Alternatively, the commissure attachment features, the connecting struts, the stent or any combination may undergo a coldworking process. In cases where the commissure attachment features, the connecting struts, and the stent are formed of different materials, any of these processes may be performed on only some of the components (e.g., on only the commissure attachment feature and/or the connecting struts) before they are collectively attached to form a stent. Alternatively, the treatments may be performed on all of the components. In some examples, the amount of coldworking is a gradient extending from one end of the commissure attachment feature to the other (e.g., the amount of coldworking at the distal end of the commissure attachment feature is greater or less than the amount of coldworking at the proximal end of the commissure attachment feature).

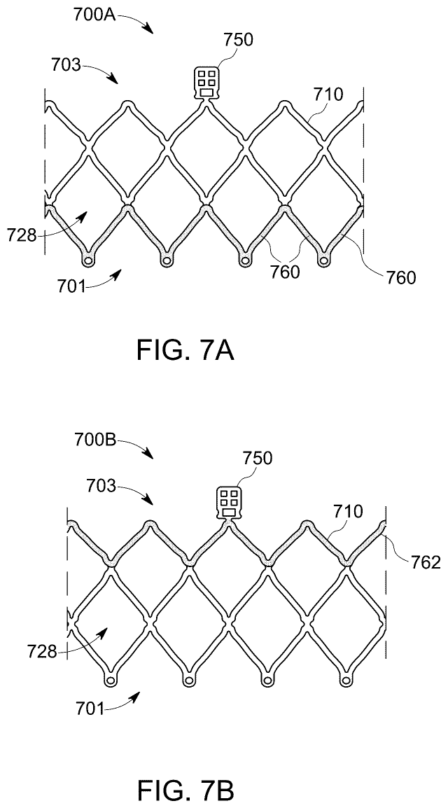

In addition to modifying the commissure attachment feature or commissure region, similar techniques as described above may applied to the inflow end or outflow end. FIG. 7A illustrates a stent 700A having an inflow end 701 and an outflow end 703, the stent having struts 710 that form cells 728. In this example, the proximal-most struts 760 have been modified to improve deflection and enable flaring in order to improve anchoring of the stent within the native vale annulus. In some examples, struts 760 may be modified using any of the techniques previously described (e.g., by formed the struts 760 of a different, more flexible secondary material and coupled to the remainder of the stent as described with reference to FIGS. 4A-D, by providing them with a thinner cross-section or thickness as described with reference to FIGS. 5A-D, or by undergoing a cold-working or annealing process or treatment to modify physical parameter(s) and create a modified microstructure as described with reference to FIG. 6).

Likewise, in FIG. 7B, a stent 700B is shown having an inflow end 701 and an outflow end 703, and struts 710 that form cells 728. In this example, the distal-most struts 762 have been modified to improve deflection and enable flaring. In some examples, struts 762 may undergo similar treatments or be formed in similar configurations to those previously described to improve deflection to improve laminar flow, minimize the vortex and turbulent flow through the stent and beyond, and potentially minimize thrombus potential.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims. Further, it should be understood that different embodiments described herein may be combined with other embodiments described herein to achieve the benefits of both embodiments.

1. A prosthetic heart valve, comprising:
a collapsible and expandable stent having an inflow end and an outflow end, and a plurality of struts defining a plurality of cells, the stent being formed of a first material;
a plurality of commissure attachment features formed of a second material that is different than the first material of the stent; and
a collapsible and expandable valve assembly including a plurality of leaflets connected to the plurality of commissure attachment features.

2. The prosthetic heart valve of claim 1, wherein the second material is more flexible than the first material.

3. The prosthetic heart valve of claim 2, wherein the second material comprises a second metallic alloy that is different from a first metallic alloy of the stent.

4. The prosthetic heart valve of claim 2, wherein the second material comprises a polymer.

5. The prosthetic heart valve of claim 1, further comprising at least one connecting strut for coupling each of the plurality of commissure attachment features to the stent, the at least one connecting strut comprising the second material.

6. The prosthetic heart valve of claim 5, wherein the at least one connecting strut comprises two connecting struts arranged in a V-shape and being connected to an uppermost row of cells disposed adjacent the outflow end of the stent.

7. The prosthetic heart valve of claim 5, wherein the at least one connecting strut comprises two connecting struts arranged in a V-shape and being connected to a lowermost row of cells disposed adjacent the inflow end of the stent.

8. The prosthetic heart valve of claim 5, wherein the at least one connecting strut is bonded or welded to the collapsible and expandable stent.

9. A prosthetic heart valve, comprising:
a collapsible and expandable stent having an inflow end and an outflow end, and a plurality of struts defining a plurality of cells, the stent having a first thickness;
a plurality of commissure attachment features having a second thickness that is less than the first thickness of the stent; and
a collapsible and expandable valve assembly including a plurality of leaflets connected to the plurality of commissure attachment features.

10. The prosthetic heart valve of claim 9, wherein the collapsible and expandable stent and the plurality of commissure attachment features gradually decrease from the first thickness to the second thickness.

11. The prosthetic heart valve of claim 9, wherein the collapsible and expandable stent and the plurality of commissure attachment features linearly decrease from the first thickness to the second thickness.

12. The prosthetic heart valve of claim 9, wherein the collapsible and expandable stent and the plurality of commissure attachment features include a transitioning step between the first thickness and the second thickness.

13. The prosthetic heart valve of claim 9, wherein the first thickness is twice the second thickness.

14. The prosthetic heart valve of claim 9, wherein the ratio of the first thickness to the second thickness is between 1.5:1 and 3:1.

15. A method of forming a prosthetic heart valve, comprising:
providing a collapsible and expandable stent having an inflow end and an outflow end, and a plurality of struts defining a plurality of cells;
forming a plurality of commissure attachment features, the plurality of commissure attachment features being more flexible than the collapsible and expandable stent; and
coupling a collapsible and expandable valve assembly including a plurality of leaflets connected to the plurality of commissure attachment features.

16. The method of claim 15, wherein forming a plurality of commissure attachment features comprises forming a plurality of commissure attachment features from a second material that is different than a first material of the stent, and further comprising the step of coupling the plurality of commissure attachment to the collapsible and expandable stent.

17. The method of claim 15, wherein forming a plurality of commissure attachment features further comprises reducing a wall thickness of the plurality of commissure attachment features to be less than a wall thickness of the stent.

18. The method of claim 15, wherein reducing a wall thickness of the plurality of commissure attachment features comprises machining, targeted grit blasting or electropolishing the plurality of commissure attachment features.

19. The method of claim 15, further comprising the step of cold working the plurality of commissure attachment features to increase flexibility.

20. The method of claim 15, further comprising the step of annealing the plurality of commissure attachment features to increase flexibility.
